(19) United States
(12) Patent Application Publication
(10) Pub. No.: US 2006/0129219 A1
(43) Pub. Date: Jun. 15, 2006

(54) Tool for placement of dual angioplasty wires in the coronary sinus vasculature and method of using the same

(76) Inventor: Paul A Kurth, Santa Barbara, CA (US)

Correspondence Address: Daniel L Dawes;Myers Dawes Andras & Sherman, 19900 MacArthur Boulevard, Suite 1150, Irvine, CA, 92612, US

(21) Appl. No.: 10/526,288

(22) PCT Filed: Aug. 28, 2003
(86) PCT No.: PCT/US03/27291

§ 371 Date: Oct. 11, 2005

Related U.S. Application Data
(60) Provisional application No. 60/408,385, filed on Sep. 5, 2002.

(51) Int. Cl. A61N 1/05

(52) U.S. Cl. 607/119

(57) ABSTRACT

An apparatus for facilitating implantation of a pacemaker lead into the vasculature of the coronary sinus of a human heart comprises a flexible guidewire for endovascular disposition; a flexible elongate tool having at least one lumen defined therethrough for telescopic disposition of the flexible guidewire therein, the elongate tool being telescopically disposed over the flexible guidewire; and a straightening wire for telescopic disposition into the elongate tool, the straightening wire for urging the elongate tool into a more straightened shape, which is sufficiently straightened to provide increased ease of telescopic disposition of the pacemaker lead over either the first or straightening wire. In one embodiment the elongate tool includes at least two lumens defined therein to allow simultaneous telescopic disposition of the first and straightening wire therein. It is also contemplated that the elongate tool may have only lumen defined therein.

### RELATED APPLICATIONS

The present application is related to U.S. Provisional Patent Application Ser. No. 60/408,385, filed on Sep. 5, 2002, which is incorporated herein by reference and to which priority is claimed pursuant to 35 USC 119.

### BACKGROUND OF THE INVENTION

1. Field of the Invention

The invention relates to the field of pacemaker lead implantation methodologies and apparatus and in particular to pacemaker leads implanted into the vasculature of the coronary sinus of the human heart.

2. Description of the Prior Art

Cardiac pacemakers for treating bradycardia commonly employ pacing leads for connecting an electrical pulse generator to excitable cardiac tissue, usually within the heart's right ventricle. Such leads have one or more electrodes proximate the distal end thereof and also commonly employ tines located just distal of the tip electrode for holding that electrode in contact with endocardial tissue in the right ventricle. The tines engage the trabeculae, resisting movement of the lead tip due to body movement and/or contractions of the heart muscle itself.

Cardiac stimulation can have a beneficial effect in treating patients suffering from congestive heart failure (CHF). By properly controlling the AV interval of the pacemaker a sick heart may be made to pump more efficiently. Pacing therapy for the treatment of CHF, however, often requires the ability to stimulate the left ventricle, either alone or in conjunction with right ventricular stimulation. Previous methods for achieving left ventricular pacing require placement of an epicardial lead, via thoracotomy or a thoracoscopic approach. Because of the usual poor condition of CHF patients, both of these procedures are “high risk” due to the trauma of the surgery itself and the need for general anesthesia. To obviate the need for a thoracotomy, left ventricular access (LVA) leads have been developed that may be introduced through the coronary sinus and then advanced through the coronary veins so that the lead's stimulating electrode can be positioned on the surface of the left ventricle near the apex of the heart.

Those skilled in the art knowing the anatomical configuration and dimensions of the coronary veins on the heart can appreciate that a lead to be routed therethrough must be of a relatively small diameter as compared to a conventional pacing lead adapted for placement in the right ventricle. Heart motion and respiratory motion as well as blood blow or other body movement are typical mechanisms for lead dislodgment. These problems are deemed to be more acute in CHF patients due to the dilated condition of CHF hearts and general diseased state of the tissue.

It can be seen, then, that a need exists for some type of method and apparatus for advancing a pacing lead through the coronary sinus and thence through a coronary vein on the heart and positioning the electrode at a desired site notwithstanding heart motion, respiratory motion blood flow and other body movement.

The placement of leads into the vasculature of the coronary sinus is often difficult, even when using preimplanted angioplasty guidewires, because the guidewire is necessarily flexible, light and conforms to the tortuous paths of the sinus vasculature. Thus, a pacemaker lead using the angioplasty guidewire as a means for implantation can easily become stuck or jammed on a particularly sharp or complex bend or curved path.

### BRIEF SUMMARY OF THE INVENTION

The invention is an apparatus for facilitating implantation of a pacemaker lead into the vasculature of the coronary sinus of a human heart comprising a flexible wire, guidewire, or guide for endovascular disposition into the vasculature of the coronary sinus; a flexible elongate tool, catheter or introducer having at least one lumen defined therethrough for telescopic disposition of the flexible wire therein, the tool being telescopically disposed over the flexible wire; and a straightening wire for telescopic disposition into the tool, the straightening wire for urging the vasculature into a more straightened configuration, which is sufficiently straightened to provide increased ease of telescopic disposition of the pacemaker lead over either the flexible wire or straightening wire into the vasculature of the coronary sinus of the human heart.

The tool includes at least two lumens defined therein to allow simultaneous telescopic disposition of the flexible wire and straightening wire therein.

The straightening wire is stiffer than the flexible wire and induces further straightening of the vasculature when it is disposed therein.

In the embodiment where the tool has a single lumen defined therein, and where the straightening wire is stiffer than the flexible wire and induces straightening of the vasculature when it is disposed therein, the flexible wire is removed from the single lumen of the tool to allow telescopic disposition of the straightening wire therein.

The tool has a predetermined stiffness greater than the flexible wire and telescopic disposition of the tool over the flexible wire induces straightening of the vasculature into which it is disposed.

The flexible wire is utilized for the telescopic disposition of the pacemaker lead into the vasculature of the coronary sinus of the human heart after removal of the tool.

The invention is also defined as a kit for facilitating implantation of a pacemaker lead into the vasculature of the coronary sinus of a human heart comprising a pacemaker lead; a catheter for providing a first measure of vascular straightening; a flexible guide wire for endovascular ready disposition into the vasculature of the coronary sinus and for guiding the catheter into the vasculature of the coronary sinus of the human heart; and a straightening wire for telescopic disposition into the catheter. The straightening wire urges the vasculature of the coronary sinus into a more straightened shape and retains the vasculature in the more straightened shape when the catheter is removed, which vasculature is sufficiently straightened by the inducement of the straightening wire to provide increased ease of telescopic disposition of the pacemaker lead over either the flexible guide wire or the straightening wire into the vasculature of the coronary sinus of the human heart.

The invention is also a method for facilitating implantation of a pacemaker lead into the vasculature of the coronary sinus of a human heart comprising the steps of: disposing a flexible guide into the vasculature of the coronary sinus; telescopically disposing an elongate tool on the flexible guide, the elongate tool providing a first measure of vascular straightening; telescopically disposing a straightening wire into the elongate tool, the straightening wire urging the elongate tool and hence the vasculature of the coronary sinus into a more straightened shape; removing the elongate tool from the vasculature of the coronary sinus while leaving straightening wire in position to retain the vasculature of the coronary sinus in the more straightened shape; and telescopically disposing a pacemaker lead over either the straightening wire or the flexible guide.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a diagrammatic side cross sectional view of a tortuous vessel in the coronary sinus vasculature of the human heart with a flexible guidewire disposed therein.

FIG. 2 is a perspective end view of the elongate tool of the invention with two lumens defined therein.

FIG. 3 is a diagrammatic side cross sectional view of a tortuous vessel in the coronary sinus vasculature of the human heart with the elongate tool of FIG. 2 telescopically disposed over the wire of FIG. 1.

FIG. 4 is a diagrammatic side cross sectional view of a tortuous vessel in the coronary sinus vasculature of the human heart with a pacemaker lead telescopically disposed over a stiffer or straightening, or flexible guide wire.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

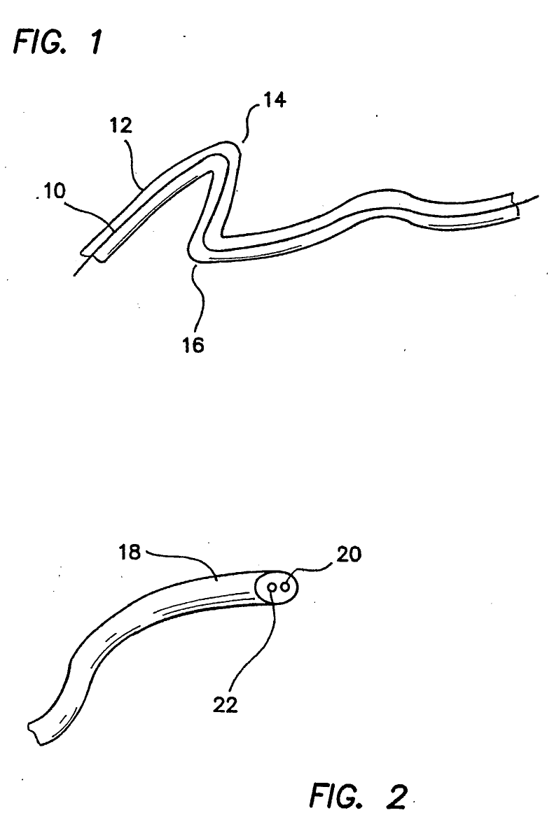

According to the invention as diagrammatically shown in FIG. 1 an angioplasty wire or wire-like device 10 is disposed by conventional means into a highly curved portion of the vasculature 12, which is illustrated as being located in the vasculature of the coronary sinus. Wire 10 is sufficiently flexible to navigate the tortuous bens 14, 16 in the vasculature of the coronary sinus. However, the curvature of wire 10 is such that if a pacemaker lead were telescopically disposed on wire 10, the pacemaker lead would likely encounter difficulties in navigating bends 14 and/or 16.

An elongate tool or catheter 18 diagrammatically depicted in FIG. 2 in enlarged scale relative to FIG. 1 is provided with two parallel longitudinal lumens 20 and 22. Tool 18 is flexible and only slightly larger than wire 10. Tool 18 may typically have an outer diameter of 2 to 4 French, while wire 10 is a stainless steel angioplasty wire in the range of 0.014 inch to 0.018 inch in diameter. Tool 18 is flexible and soft enough to pass through bends 14 and 16 in the vasculature of the coronary sinus without difficulty. Tool 18 may further be provided with a lubricious coating to facilitate ease of disposition into the vasculature. The size or diameter of tool 18 may be only slightly smaller than the pacemaker lead which it precedes. For example, tool 18 may be made from an extruded polymeric material, such as Pebax®, a trademark of Ato Chimie of Courbevoie (Hauts-de-Seine), France.

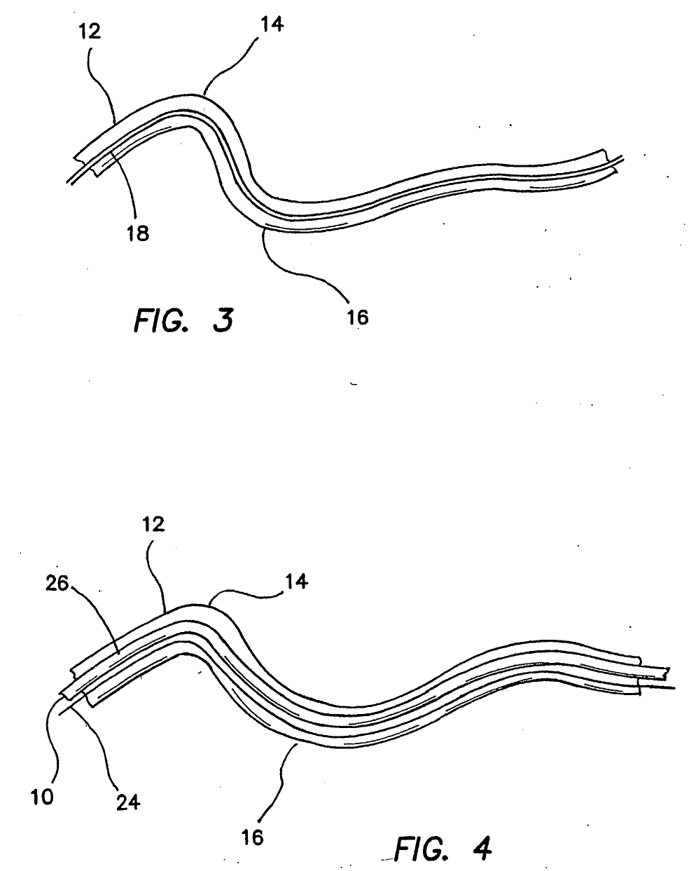

Wire 10 is telescopically disposable in one of the lumens 20, 22. Thus, once wire 10 is implanted, tool 18 is then easily telescopically disposed over wire 10 along its length as shown in FIG. 3. Because tool 18 has a slightly larger diameter than wire 10, it is slightly stiffer, but not so much more stiffer that it cannot be easily guided on wire 10. However, its slightly greater stiffness causes the vasculature 12 in which tool 18 is implanted to straighten to a degree as shown by a comparison of the shape of vasculature 12 at bends 14 and 16 in the depictions compared between FIG. 1 and FIG. 3. While tool 18 is easily guided by wire 10, its stiffness is greater so that it tends to straighten the vasculature somewhat, while the flexibility of wire 10 is such that wire 10 has no substantial effect on the shape or straightening of the vasculature.

With tool 18 implanted a second even stiffer wire or wire-like device 24 is then telescopically disposed in the second unused lumen 20 or 22 so that tool 18 is now in place with two wires 10 and 24 disposed therein. It is also contemplated that wire 10 could be removed and wire 24 inserted through the same lumen, although this is not the preferred embodiment. The invention contemplates both single and multiple lumened tools or catheters. When a single lumen is provided, the guidewire 10 and stiffening second wire 24 or straightening means would be exchanged, and then the pacemaker telescopically disposed over the second wire.

Typically, wire 24 is slightly heavier or stiffer than wire 10, so that wire 10 is easily implanted in the first instance in a tortuous vascular path, the vascular path is straightened somewhat by tool 18 and then wire 24 implanted, which wire 24 would otherwise be difficult to implant in a tortuous path if attempted first. After wires 10 and 24 are implanted, tool 18 is removed leaving wires 10 and 24 in place.

At this point vasculature 12 is held in the slightly straightened configuration of FIG. 3 even with tool 18 removed, because the presence of wire 24 is sufficient to maintain a degree of vasculature straightening. A pacemaker lead 26 is then telescopically disposed over one of the wires 10 or 24 in vasculature 12 as depicted in FIG. 4. In the preferred embodiment guidewire 10 is used to implant lead 26, since wire 24 is in close contact or subject to higher stress loads at many points in the vasculature, while wire 10 remains in a more free or floating condition in the vasculature. Wire 24 is left in place during implantation of lead 26 to maintain the vasculature straightening. Because vasculature 12 has been at least slightly straightened, pacemaker lead 26 on guidewire 10 can now successfully navigate bends 14 and 16. After successful implantation of lead 26, wires 10 and 24 are both removed and further procedures such as anchoring and further movement in the vasculature with lead 26 continue in a conventional manner.

It is to be understood that in addition to the foregoing methodology that the tool 18 may be disposed first into the vascular system as far as it may be possible, which could either reach near or to the implantation site or only be possible to the catheter take-off area, which may be the opening of the coronary sinus or the larger veins of the coronary sinus leading to the branching veins of the coronary sinus system. For this purpose the distal end of tool 18 may be curved or curvable to allow its steerability in the coronary sinus system. Wires 10 and then wire 24 in that order are then telescopically disposed into tool 18 and extended from the end of tool 18 until reaching the implantation site. At this point it may be possible to advance tool 18 to or near the implantation site, if not already so positioned, due to the vascular straightening achieved by wire 24. Tool 18 is removed and lead 26 is disposed over wire 10 or even wire 24 to or near the implantation site. Wires 10 and 24 are removed after lead 26 is implanted or anchored into position.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

We claim:

1. An apparatus for facilitating implantation of a pacemaker lead into the vasculature of the coronary sinus of a human heart comprising:
a flexible wire for endovascular disposition into the vasculature of the coronary sinus;
a flexible elongate tool having at least one lumen defined therethrough for telescopic disposition of the flexible wire therein, the tool being telescopically disposed over the flexible wire; and
a straightening wire for telescopic disposition into the tool, the straightening wire for urging the vasculature into a more straightened configuration, which is sufficiently straightened to provide increased ease of telescopic disposition of the pacemaker lead over either the flexible wire or straightening wire into the vasculature of the coronary sinus of the human heart.

2. The apparatus of claim 1 where the tool includes at least two lumens defined therein to allow simultaneous telescopic disposition of the flexible wire and straightening wire therein.

3. The apparatus of claim 2 where the straightening wire is stiffer than the flexible wire and induces further straightening of the vasculature when it is disposed therein.

4. The apparatus of claim 1 where the tool has a single lumen defined therein, where the straightening wire is stiffer than the flexible wire and induces straightening of the vasculature when it is disposed therein, the flexible wire being removed from the single lumen of the tool to allow telescopic disposition of the straightening wire therein.

5. The apparatus of claim 1 where the tool has a predetermined stiffness greater than the flexible wire and where telescopic disposition of the tool over the flexible wire induces straightening of the vasculature into which it is disposed.

6. The apparatus of claim 2 where the flexible wire is utilized for the telescopic disposition of the pacemaker lead into the vasculature of the coronary sinus of the human heart after removal of the tool.

7. A kit for facilitating implantation of a pacemaker lead into the vasculature of the coronary sinus of a human heart comprising:
a catheter for providing a first measure of vascular straightening;
a flexible guide wire for endovascular ready disposition into the vasculature of the coronary sinus and for guiding the catheter into the vasculature of the coronary sinus of the human heart; and
a straightening wire for telescopic disposition into the catheter, the straightening wire for urging the vasculature of the coronary sinus into a more straightened shape and for retaining the vasculature in the more straightened shape when the catheter is removed, which vasculature is sufficiently straightened by the inducement of the straightening wire to provide increased ease of telescopic disposition of the pacemaker lead over either the flexible guide wire or the straightening wire into the vasculature of the coronary sinus of the human heart.

8. The kit of claim 7 where the elongate means includes at least two lumens defined therein to allow simultaneous telescopic disposition of the flexible guide wire and the straightening wire therein.

9. The kit of claim 8 where the straightening wire is stiffer than the catheter.

10. The kit of claim 7 where the catheter has a single lumen defined therein, where the straightening wire is stiffer than the catheter, the flexible guide wire being removed from the single lumen to allow telescopic disposition of the straightening wire therein.

11. The kit of claim 7 where the catheter has a predetermined stiffness greater than the flexible guide wire.

12. The kit of claim 8 where either the flexible guide wire or the straightening wire provides guidance for the telescopic disposition of the pacemaker lead into the vasculature of the coronary sinus of the human heart.

13. The kit of claim 8 where either the flexible guide wire provides guidance for the telescopic disposition of the pacemaker lead into the vasculature of the coronary sinus of the human heart.

14. A method for facilitating implantation of a pacemaker lead into the vasculature of the coronary sinus of a human heart comprising:
disposing a flexible guide into the vasculature of the coronary sinus;
telescopically disposing an elongate tool on the flexible guide, the elongate tool providing a first measure of vascular straightening;
telescopically disposing a straightening wire into the elongate tool, the straightening wire urging the elongate tool and hence the vasculature of the coronary sinus into a more straightened shape;
removing the elongate tool from the vasculature of the coronary sinus while leaving straightening wire in position to retain the vasculature of the coronary sinus in the more straightened shape; and
telescopically disposing a pacemaker lead over either the straightening wire or the flexible guide.

15. The method of claim 14 where the elongate means includes at least two lumens defined therein so that the elongate tool is telescopically disposed over the flexible guide and the straightening wire is telescopically disposed into the elongate tool.

16. The method of claim 15 where the straightening wire is stiffer than the elongate tool and induces further straightening of the vasculature into which it is disposed.

17. The method of claim 14 where the elongate tool has a single lumen defined therein and where the straightening wire is stiffer than the elongate tool and induces further straightening of the vasculature into which the straightening wire is disposed, the flexible guide being removed from the lumen to allow telescopic disposition of the straightening wire therein.

18. The method of claim 14 where the elongate tool has a predetermined stiffness greater than the flexible guide, and where telescopic disposition of the elongate tool over the flexible guide induces straightening of the vasculature into which it is disposed.

19. The method of claim 15 where either the flexible guide or the straightening wire provides guidance for the telescopic disposition of the pacemaker lead into the vasculature of the coronary sinus of the human heart.

20. The method of claim 15 where either the flexible guide provides guidance for the telescopic disposition of the pacemaker lead into the vasculature of the coronary sinus of the human heart.
